(19) United States
(12) Patent Application Publication
(10) Pub. No.: US 2020/0315549 A1
(43) Pub. Date: Oct. 8, 2020

(54) ATTENTION ALERTING SYSTEM, INFORMATION PROCESSING DEVICE, INFORMATION PROCESSING METHOD, AND NON-TRANSITORY RECORDING MEDIUM IN WHICH PROGRAM IS STORED

(71) Applicant: OMRON HEALTHCARE Co., Ltd., Kyoto (JP)

(72) Inventors: Hiroshi USUI, Kyoto (JP); Hironori SATO, Kyoto (JP); Kosuke INOUE, Kyoto (JP); Yasushi MATSUOKA, Kyoto (JP)

(21) Appl. No.: 16/910,350

(22) Filed: Jun. 24, 2020

Related U.S. Application Data
(63) Continuation of application No. PCT/JP2018/046243, filed on Dec. 17, 2018.

(30) Foreign Application Priority Data
Dec. 27, 2017 (JP) 2017-252519

(51) Int. Cl. A61B 5/00; A61B 5/11; G16H 40/63

(52) U.S. Cl. CPC A61B 5/746; A61B 5/7475; A61B 5/742; A61B 5/681; A61B 5/1112; A61B 5/0235; A61B 5/002; G16H 40/63; A61B 2562/0219; A61B 2562/0247; A61B 5/0004

(57) ABSTRACT

An information processing device according to one aspect of the present invention includes an acquisition unit configured to acquire user data including physiological indicator data that are measurement data relating to a physiological indicator of a user and position data that are measurement data relating to a position of the user, a time identification unit configured to identify a time when fluctuations in the physiological indicator satisfying a predetermined condition have occurred, based on the physiological indicator data, a position identification unit configured to identify a position where the user has been present at the identified time, based on the position data, and an observed region determination unit configured to determine a local region including the identified position as an observed region for performing an attention alert relating to an occurrence of fluctuations in the physiological indicator.

### CROSS-REFERENCE TO RELATED APPLICATIONS

This is a continuation of International Application 2017-252519, with an international filing date of Dec. 27, 2017, and International Application PCT/JP2018/046243, with an international filing date of Dec. 17, 2018, filed by applicant, the disclosure of which is hereby incorporated by reference in its entirety.

### TECHNICAL FIELD

The present invention relates to an attention alerting system for performing an attention alert relating to biological information.

### BACKGROUND ART

A device that monitors a physiological indicator of a user such as a blood pressure value or a pulse rate, and issues a warning in response to significant fluctuations in the physiological indicator has been known. For example, JP 2016-154578 A discloses a wearable electronic appliance that acquires biological information about a user, and notifies the user that an abnormality has occurred (for example, a pulse rate has become zero) in the biological information when detecting that the abnormality has occurred.

The electronic appliance disclosed in JP 2016-154578 A can detect that an abnormality has occurred in biological information, but cannot predict and notify that an abnormality is likely to occur in biological information.

### SUMMARY OF INVENTION

The present invention has been made in view of the circumstance described above, and an object thereof is to provide an attention alerting system capable of alerting a user that the user is under a situation where significant fluctuations in a physiological indicator are likely to occur, and an information processing device, an information processing method, and a non-transitory recording medium in which a program is stored, which allow the user to be alerted that the user is under the situation where the significant fluctuations in the physiological indicator are likely to occur.

The present invention adopts the following aspect in order to solve the problem described above.

An information processing device according to one aspect of the present invention includes an acquisition unit configured to acquire user data including physiological indicator data that are measurement data relating to a physiological indicator of a user and position data that are measurement data relating to a position of the user, a time identification unit configured to identify a time when fluctuations in the physiological indicator satisfying a predetermined condition have occurred, based on the physiological indicator data, a position identification unit configured to identify a position where the user is present at the identified time, based on the position data, and an observed region determination unit configured to determine a local region including the identified position as an observed region for performing an attention alert relating to an occurrence of fluctuations in the physiological indicator.

In the present specification, the physiological indicator refers to an indicator relating to biological information about a human being. The biological information is information that can be acquired from a human body, and is, for example, blood pressure, a pulse wave, a pulse, a heart rate, and the like. The physiological indicator is, for example, a blood pressure value, a pulse rate, a heart rate, a stress value, and the like. The stress value represents a degree to which a user is under mental stress, and is calculated based on the pulse rate or the heart rate, for example.

When a target user revisits a place where the physiological indicator of the target user himself/herself has significantly fluctuated in the past, the physiological indicator is likely to significantly fluctuate again. Similarly, when the target user also visits a place where the physiological indicator of another user has significantly fluctuated, the physiological indicator of the target user is likely to significantly fluctuate. Therefore, such a place can be considered as a situation where the significant fluctuations in the physiological indicator are likely to occur.

In the configuration described above, a position where a user (a target user or another user) is located when the physiological indicator of the user significantly fluctuates is identified, and a local region including the identified position is determined as an observed region for performing an attention alert relating to an occurrence of the significant fluctuations in the physiological indicator. For example, when the observed region is set in a user terminal carried by the target user, the user terminal can alert the target user that the target user is under the situation where the significant fluctuations in the physiological indicator are likely to occur when the target user is present in the observed region.

The information processing device according to the aspect described above may further include a time range determination unit configured to determine a time range for making a notification, based on the identified time, and the observed region determination unit associates the observed region with the determined time range.

According to the configuration described above, it is possible to make a notification when the target user enters the observed region at a time within the associated time range. As a result, the target user can be prevented from being unnecessarily alerted when the significant fluctuations in the physiological indicator tend to occur during a specific time period in the observed region.

In the information processing device according to the aspect described above, the user data may further include attribute information representing an attribute of the user, and the observed region determination unit associates the observed region with an attribute group based on the attribute.

According to the configuration described above, an attention alert can be notified to the target user with, as a target, the observed region determined based on the user data relating to the user belonging to the same attribute group (for example, age and/or gender) as that of the target user. The significant fluctuations in the physiological indicator of the target user are more likely to occur in the observed region associated with the user belonging to the same attribute group as that of the target user than in the observed region associated with the user belonging to another attribute group. Therefore, the target user can be alerted when the significant fluctuations in the physiological indicator are more likely to occur.

In the information processing device according to the aspect described above, the user data may further include identification information for identifying the user, and the observed region determination unit associates the observed region with the identification information.

According to the configuration described above, it is possible to identify whether the observed region is determined based on the user data relating to the target user or is determined based on the user data relating to another user. The significant fluctuations in the physiological indicator of the target user are more likely to occur in the observed region associated with the target user than in the observed region associated with the another user. When the target user enters the observed region associated with the target user himself/herself, an attention alerting method can be changed between the observed region associated with the target user and the observed region associated with the another user, such as by performing a clearer attention alert and the like.

The information processing device according to the aspect described above may further include a transmission unit configured to transmit observed region information representing the observed region to a plurality of user terminals, and the acquisition unit may acquire the user data relating to a plurality of users from the plurality of user terminals.

In the configuration described above, the observed region is determined based on not only the user data relating to the target user but also the user data relating to another user. In this way, it is also possible to perform an attention alert in a place and the like where the target user visits for the first time.

In the information processing device according to the aspect described above, the physiological indicator may be a blood pressure value, the condition may include a condition that the blood pressure value rises by greater than or equal to a predetermined value within a predetermined period, and the time identification unit identifies a time when fluctuations in blood pressure value satisfying the condition have occurred.

According to the configuration described above, the target user can be alerted that the target user is under the situation where rapid fluctuations in blood pressure are likely to occur.

The present invention can provide the attention alerting system capable of alerting a user that the user is under a situation where significant fluctuations in a physiological indicator are likely to occur, and the information processing device, the information processing method, and the non-transitory recording medium in which the program is stored, which allow the user to be alerted that the user is under the situation where the significant fluctuations in the physiological indicator are likely to occur.

### BRIEF DESCRIPTION OF DRAWINGS

FIG. 1 is a block diagram illustrating an attention alerting system according to an embodiment.

FIG. 2 is a diagram illustrating a system configuration of an attention alerting system according to the embodiment.

FIG. 3 is a block diagram illustrating one example of a hardware configuration of a biological information processing device illustrated in FIG. 2.

FIG. 4 is a block diagram illustrating one example of a hardware configuration of a portable terminal illustrated in FIG. 2.

FIG. 5 is a block diagram illustrating one example of a hardware configuration of a blood pressure monitor illustrated in FIG. 2.

FIG. 6 is a block diagram illustrating one example of a software configuration of the biological information processing device illustrated in FIG. 2.

FIG. 7 is a diagram illustrating an example of a method for detecting rapid blood pressure fluctuations according to the embodiment.

FIG. 8 is a diagram illustrating a map in which an observed region according to the embodiment is set.

FIG. 9 is a block diagram illustrating one example of a software configuration of the portable terminal illustrated in FIG. 2.

FIG. 10 is a block diagram illustrating one example of a software configuration of the blood pressure monitor illustrated in FIG. 2.

FIG. 11 is a flowchart illustrating a procedure of determining an observed region according to the embodiment.

FIG. 12 is a flowchart illustrating a procedure of an attention alert according to the embodiment.

### DESCRIPTION OF EMBODIMENTS

Hereinafter, an embodiment of the present invention will be described with reference to the drawings.

### APPLICATION EXAMPLE

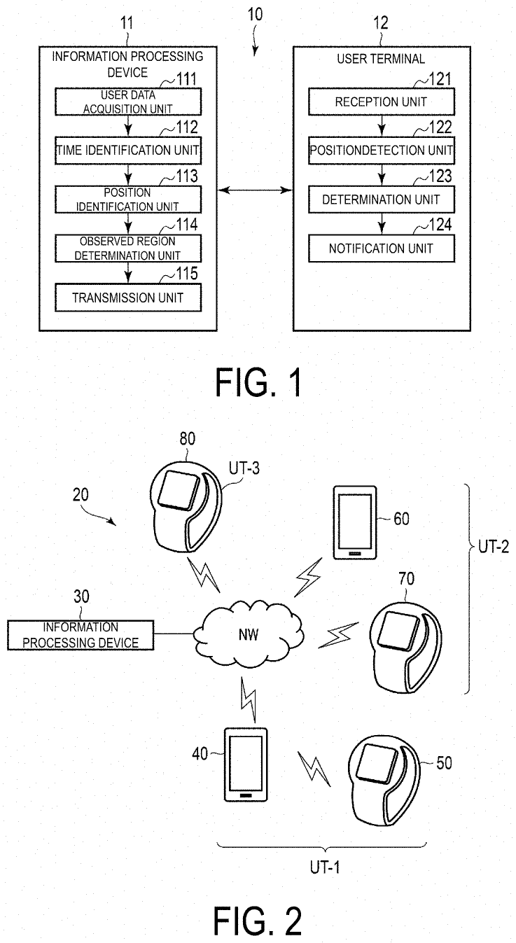

One application example of the present invention will be described with reference to FIG. 1. FIG. 1 illustrates an attention alerting system 10 according to one embodiment of the present invention. The attention alerting system 10 includes an information processing device 11 and a user terminal 12. The information processing device 11 can communicate with the user terminal 12.

The information processing device 11 determines an observed region that is a geographical region for performing an attention alert relating to an occurrence of significant fluctuations in a physiological indicator. The physiological indicator is, for example, a blood pressure value, a pulse rate, a heart rate, a stress value, and the like. The significant fluctuations in the physiological indicator refer to fluctuations in a physiological indicator that is likely to affect health of a user. Hereinafter, the significant fluctuations in the physiological indicator may be referred to as an event. Examples of the event include a phenomenon in which a blood pressure value rapidly rises, a phenomenon in which a blood pressure value rapidly falls, a rapid rise in a stress value, and the like. It has been known that a repeated occurrence of a rapid blood pressure rise accelerates onset of a brain disease or a cardiovascular disease.

The information processing device 11 includes a user data acquisition unit 111, a time identification unit 112, a position identification unit 113, an observed region determination unit 114, and a transmission unit 115.

The user data acquisition unit 111 acquires user data relating to one or more users. The user data relating to each user include physiological indicator data that are measurement data relating to a physiological indicator of the user and position data that are measurement data relating to a position of the user. The physiological indicator data are data representing a transition (a change over time) of a physiological indicator of the user, and include a measurement result of a physiological indicator associated with time information. The position data are data representing a transition of a position of the user, and includes a measurement result of a position associated with time information.

The time identification unit 112 identifies, based on the physiological indicator data, a time when fluctuations in a physiological indicator satisfying a predetermined condition have occurred. The predetermined condition is a condition for detecting an occurrence of an event. The condition will be described below.

The position identification unit 113 identifies, based on the position data, a position where the user has been present at the time identified by the time identification unit 112. The position identified by the position identification unit 113 represents a position where an event has actually occurred.

The observed region determination unit 114 determines, as an observed region, a local region including the position identified by the position identification unit 113. For example, the observed region determination unit 114 determines, as an observed region, a region within a circle having a certain radius centered in the position identified by the position identification unit 113.

The transmission unit 115 transmits, to the user terminal 12, observed region information representing the observed region determined by the observed region determination unit 114.

The user terminal 12 monitors a position of a target user (a user carrying the user terminal 12), and alerts the target user that the target user is under a situation where an event is likely to occur when the target user is present within the observed region. The target user may be one user or one of the plurality of users described above, and may be different from the one or the plurality of users described above. The user terminal 12 includes a reception unit 121, a position detection unit 122, a determination unit 123, and a notification unit 124.

The reception unit 121 receives the observed region information from the information processing device 11. The position detection unit 122 detects a position of the user terminal 12, namely, a position of the target user. The determination unit 123 determines whether or not the position detected by the position detection unit 122 is located in the observed region indicated by the observed region information received by the reception unit 121. The notification unit 124 outputs a notification for an attention alert in response to the determination by the determination unit 123 that the detected position is located in the observed region. For example, when the target user moves and enters the observed region, the determination unit 123 provides, to the notification unit 124, a signal representing that the target user has entered the observed region, and the notification unit 124 receives this signal and displays a message for the attention alert on a display device.

In the attention alerting system 10 including the configuration described above, a position where an event has actually occurred is identified, and a local region including the identified position is determined as an observed region for performing the attention alert relating to the occurrence of the event. Then, when it is detected that a target user has entered the observed region, the attention alert is performed on the target user. In this way, when the target user is under a situation where the event is likely to occur, the user can be alerted. The target user is alerted and can take an action (for example, a deep breath and the like) for preventing an occurrence of the event.

Next, the attention alerting system according to the embodiment mentioned above will be more specifically described.

### CONFIGURATION EXAMPLE

### System Configuration

FIG. 2 illustrates a configuration example of an attention alerting system 20 according to one embodiment of the present invention. The attention alerting system 20 includes an information processing device 30 and one or a plurality of user terminals UT. The information processing device 30 and the user terminal UT are connected to a network NW such as the Internet, and thus the information processing device 30 can communicate with the user terminal UT. The information processing device 30 can include a configuration similar to that of the information processing device 11 illustrated in FIG. 1.

In the example in FIG. 2, three user terminals UT (specifically, user terminals UT-1, UT-2, and UT-3) are illustrated. Typically, each of the user terminals UT-1, UT-2, and UT-3 is used by a different user. The user terminal UT-1 includes a portable terminal 40 and a blood pressure monitor 50. The portable terminal 40 communicates with the information processing device 30 via the network NW, and directly communicates with the blood pressure monitor 50. The portable terminal 40 can include a configuration similar to that of the user terminal 12 illustrated in FIG. 1. The user terminal UT-2 includes a portable terminal 60 and a blood pressure monitor 70. The portable terminal 60 communicates with the blood pressure monitor 70 and the information processing device 30 via the network NW. The portable terminal 60 can include a configuration similar to that of the user terminal 12 illustrated in FIG. 1. The user terminal UT-3 includes a blood pressure monitor 80. The blood pressure monitor 80 communicates with the information processing device 30 via the network NW. The blood pressure monitor 80 can include a configuration similar to that of the user terminal 12 illustrated in FIG. 1. Hereinafter, the user terminal UT-1 will be mainly described.

The blood pressure monitor 50 is a wearable device worn by the user. The blood pressure monitor 50 measures a blood pressure value of the user, and generates blood pressure value data that are measurement data relating to the blood pressure value of the user. The blood pressure monitor 50 measures, for example, a blood pressure value for each heart rate. The blood pressure value includes at least one of systolic blood pressure (SBP) and diastolic blood pressure (DBP), which is not limited thereto. The blood pressure value data are data representing a transition of a blood pressure value of the user, and include a measurement blood pressure value associated with time information. The blood pressure monitor 50 is one example of a wearable biological information measuring device that measures a physiological indicator of the user. The biological information measuring device may be a wearable stress measuring instrument that measures a stress value of the user. The stress measuring instrument measures, for example, a heart rate or a pulse rate, and calculates a stress value, based on the measurement result.

Furthermore, the blood pressure monitor 50 measures a position of the blood pressure monitor 50 itself, namely, a position of the user wearing the blood pressure monitor 50, and generates position data that are measurement data relating to the position of the user. The position data are data representing a transition of a position of the user, and includes a measurement position associated with time information. The blood pressure monitor 50 transmits the physiological indicator data and the position data to the portable terminal 40.

The portable terminal 40 can be, for example, a mobile appliance such as a smart phone, a tablet PC, and a mobile phone. The portable terminal 40 receives the physiological indicator data and the position data from the blood pressure monitor 50, and manages the physiological indicator data and the position data. The portable terminal 40 transmits user data including the physiological indicator data and the position data to the information processing device 30 through the network NW. The portable terminal 40 may separately transmit the physiological indicator data and the position data to the information processing device 30.

The information processing device 30 can be, for example, a computer such as a server. The information processing device 30 collects the user data from the user terminal UT (specifically, the user terminals UT-1, UT-2, and UT-3). The information processing device 30 determines, based on the collected user data, an observed region for performing an attention alert relating to an occurrence of an event. The information processing device 30 transmits observed region information representing the observed region to the user terminal UT.

The portable terminal 40 receives the observed region information from the information processing device 30. The portable terminal 40 monitors a position of the target user, and notifies the target user that the target user is under a situation where the event is likely to occur when the target user is present within the observed region indicated by the observed region information.

### Hardware Configuration

### Information Processing Device

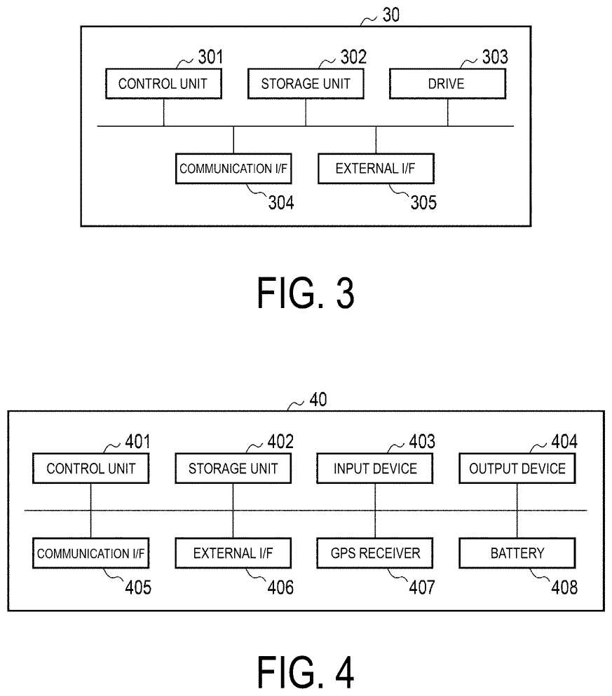

One example of a hardware configuration of the information processing device 30 according to the present embodiment will be described with reference to FIG. 3. FIG. 3 illustrates one example of the hardware configuration of the information processing device 30. In the example in FIG. 3, the information processing device 30 includes a control unit 301, a storage unit 302, a drive 303, a communication interface 304, and an external interface 305.

The control unit 301 includes a central processing unit (CPU), a random access memory (RAM), a read only memory (ROM), and the like, and controls each component according to information processing. The storage unit 302 is, for example, an auxiliary storage device such as a hard disk drive (HDD) and a semiconductor memory (for example, a solid state drive (SSD)). The storage unit 302 stores an observed region determination program executed by the control unit 301, user data received from the user terminal UT, and the like. A storage medium included in the storage unit 302 is a medium that accumulates information such as a recorded program by an electrical, magnetic, optical, mechanical, or chemical action such that a computer, a machine, and the like can read the information such as the program.

The drive 303 is a device for reading the program stored in the storage medium. A type of the drive may be appropriately selected according to a type of the storage medium. The drive 303 can be, for example, a compact disk (CD) drive, a digital versatile disk (DVD) drive, and the like. The storage medium is not limited to a disk type storage medium such as a CD, a DVD, and the like, and may be other than the disk type. Examples of the storage medium other than the disk type can include, for example, a semiconductor memory such as a flash memory. The observed region determination program described above may be stored in this storage medium. The storage medium is a medium that accumulates information such as a recorded program by an electrical, magnetic, optical, mechanical, or chemical action such that a computer, a machine, and the like can read the information such as the program. The information processing device 30 may acquire the observed region determination program described above from this storage medium.

The communication interface 304 is an interface for performing communication via the network NW. The communication interface 304 typically includes a wired communication module such as a wired local area network (LAN) module. Note that the communication interface 304 may include a wireless communication module instead of or in addition to the wired communication module. The information processing device 30 may acquire the observed region determination program described above from an external device (for example, a server (not illustrated)) through the communication interface 304.

The external interface 305 is an interface for connecting to an external appliance, and includes various kinds of terminals such as a universal serial bus (USB) port. The external appliance includes, for example, an input device such as a keyboard, an output device such as a display, a removable memory such as a USB memory, and the like.

Note that, with regard to the specific hardware configuration of the information processing device 30, it is possible to appropriately omit, replace, and add the component according to the embodiment. For example, the control unit 301 may include a plurality of processors. The information processing device 30 may be constituted by a plurality of computers. Further, in addition to the information processing device designed specifically for service to be provided, a general-purpose desktop personal computer (PC) and the like may be used as the information processing device 30.

### Portable Terminal

One example of a hardware configuration of the portable terminal 40 according to the present embodiment will be described with reference to FIG. 4. FIG. 4 illustrates one example of the hardware configuration of the portable terminal 40. In the example in FIG. 4, the portable terminal 40 includes a control unit 401, a storage unit 402, an input device 403, an output device 404, a communication interface 405, an external interface 406, a GPS receiver 407, and a battery 408.

The control unit 401 includes a CPU, a RAM, a ROM, and the like, and controls each component according to the information processing. The storage unit 402 is, for example, an auxiliary storage device such as an HDD and an SSD. The storage unit 402 stores an attention alerting program executed by the control unit 401, observed region information received from the information processing device 30, blood pressure monitor data and position data received from the blood pressure monitor 50, and the like. A storage medium included in the storage unit 402 is a medium that accumulates information such as a recorded program by an electrical, magnetic, optical, mechanical, or chemical action such that a computer, a machine, and the like can read the information such as the program.

The input device 403 and the output device 404 are achieved by, for example, a touch-screen. As a display device included in the touch-screen, for example, a liquid crystal display (LCD), an organic light emitting diode (OLED) display, and the like can be used. The input device 403 may further include a push type button, a microphone, and the like. The output device 404 may further include a speaker and the like.

The communication interface 405 is an interface for communicating with the external device. The communication interface 405 includes, for example, a wireless LAN module and a near-field wireless communication module. For example, the wireless LAN module is used for communicating with the information processing device 30 via the network NW. The near-field wireless communication module is used for directly communicating with the blood pressure monitor 50. The near-field wireless communication module is, for example, a Bluetooth (trade name) module.

The GPS receiver 407 receives a GPS signal from a plurality of GPS satellites, and outputs the received GPS signal to the control unit 401. The control unit 401 calculates, based on the GPS signal, a position of the portable terminal 40, namely, a position of the user carrying the portable terminal 40.

The battery 408 can be, for example, a rechargeable battery. The battery 408 supplies power to the control unit 401, the storage unit 402, the input device 403, the output device 404, the communication interface 405, the external interface 406, and the GPS receiver 407.

Note that, with regard to the specific hardware configuration of the portable terminal 40, it is possible to appropriately omit, replace, and add the component according to the embodiment. For example, the control unit 401 may include a plurality of processors. The portable terminal 40 may further include another sensor such as an acceleration sensor, an air pressure sensor, and an optical sensor.

### Blood Pressure Monitor

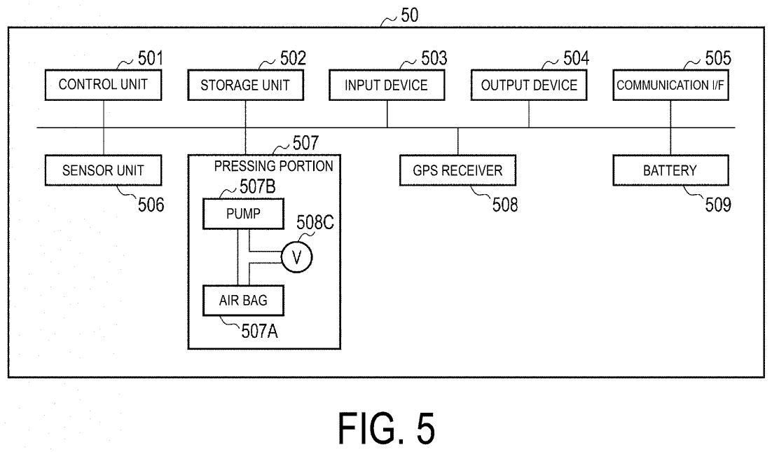

One example of a hardware configuration of the blood pressure monitor 50 according to the present embodiment will be described with reference to FIG. 5. FIG. 5 illustrates one example of the hardware configuration of the blood pressure monitor 50. The blood pressure monitor 50 illustrated in FIG. 5 measures a pressure pulse wave by a tonometry method, and calculates a blood pressure value, based on the detected pressure pulse wave. Here, the tonometry method refers to a method for pressing an artery from above a skin with appropriate pressure, forming a flat portion in the artery, and noninvasively measuring a pressure pulse wave by a pressure sensor in a balanced state between the interior and the exterior of the artery. In the tonometry method, a blood pressure value for each heart rate can be acquired.

In the example in FIG. 5, the blood pressure monitor 50 includes a control unit 501, a storage unit 502, an input device 503, an output device 504, a communication interface 505, a sensor unit 506, a pressing portion 507, a GPS receiver 508, and a battery 509.

The control unit 501 includes a CPU, a RAM, a ROM, and the like, and controls each component according to the information processing. The storage unit 502 is, for example, an auxiliary storage device such as a semiconductor memory (for example, a flash memory). The storage unit 502 stores a blood pressure measurement program executed by the control unit 501, data about a measurement result including a blood pressure value calculated by the control unit 501, and the like.

The input device 503 allows the user to input an instruction to the blood pressure monitor 50. The input device 503 provides an instruction signal according to an operation by the user to the control unit 501. The input device 503 includes, for example, a plurality of push type buttons. The output device 504 includes a display device that displays information such as a measurement result. For example, an LCD, an OLED display, and the like can be used as the display device. A touch-screen may be used as a combination of the input device 503 and the display device.

The communication interface 505 is an interface for communicating with the external device. The communication interface 505 includes a near-field wireless communication module such as a Bluetooth (trade name) module so as to communicate with the portable terminal 40, which is not limited thereto. The communication interface 505 may include another type of a wireless communication module such as a wireless LAN module. Further, the communication interface 505 may include a wired communication module.

The sensor unit 506 is disposed so as to contact a wrist in which a radial artery is present. The sensor unit 506 includes a pressure sensor array on a main surface (a surface that contacts the wrist) of the sensor unit 506, and the pressure sensor array includes a plurality of (for example, 46) pressure sensors aligned in one direction. The alignment direction of the pressure sensor is a direction that intersects a direction in which the radial artery extends in a state where the blood pressure monitor 50 is worn by the user. Each of the pressure sensors detects pressure, and generates a pressure signal indicating the detected pressure. As the pressure sensor, for example, a piezoresistive pressure sensor can be used. The pressure signal is amplified by an amplifier, is converted to a digital signal by an analog-digital converter, and is then provided to the control unit 501. A sampling frequency is, for example, 125 Hz.

The pressing portion 507 presses the sensor unit 506 against the wrist. In the tonometry method, a pressure pulse wave and blood pressure are equal under an optimum pressing condition. The pressing portion 507 includes an air bag 507A, a pump 507B that supplies air to the air bag 507A, and an exhaust valve 507C for exhausting air from the air bag 507A. When the pump 507B is driven under control by the control unit 501 so as to increase the internal pressure of the air bag 507A, the sensor unit 506 is pressed against the wrist due to the expansion of the air bag 507A. Note that the pressing portion 507 is not limited to the structure using the air bag, and may be achieved by any structure as long as the force of pressing the sensor unit 506 against the wrist can be adjusted.

In the blood pressure monitor 50, a blood pressure measurement is performed in a state where the sensor unit 506 is held in an arrangement suitable for measurement by the pressing portion 507. The control unit 501 calculates a blood pressure value, based on a pressure signal output from one pressure sensor selected from among the pressure sensors, for example. The control unit 501 stores the calculated blood pressure value in association with additional information including time information in the storage unit 502.

The GPS receiver 508 receives a GPS signal from a plurality of GPS satellites, and outputs the received GPS signal to the control unit 501. The control unit 501 calculates a position of the blood pressure monitor 50, namely, a position of the user wearing the blood pressure monitor 50, based on the GPS signal.

The battery 509 can be, for example, a rechargeable battery. The battery 509 supplies power to the control unit 501, the storage unit 502, the input device 503, the output device 504, the communication interface 505, the sensor unit 506, the pressing portion 507, and the GPS receiver 508.

Note that, with regard to the specific hardware configuration of the blood pressure monitor 50, it is possible to appropriately omit, replace, and add the component according to the embodiment. For example, the control unit 501 may include a plurality of processors. The blood pressure monitor 50 may further include another sensor such as an acceleration sensor. The blood pressure monitor 50 may further include a terminal such as a micro USB port. In this case, the blood pressure monitor 50 can be connected to an external device (for example, the portable terminal 40) by a cable such as a USB cable.

Further, the blood pressure monitor 50 is not limited to a blood pressure monitor based on the tonometry method. For example, a blood pressure monitor that detects a pulse transit time (PTT) being a transit time of a pulse wave propagating through an artery and that estimates a blood pressure value (specifically, SBP), based on the detected pulse transit time, a blood pressure monitor that optically measures a volume pulse wave and calculates a blood pressure value from the measurement result, and the like may be used as the blood pressure monitor.

### Software Configuration

### Information Processing Device

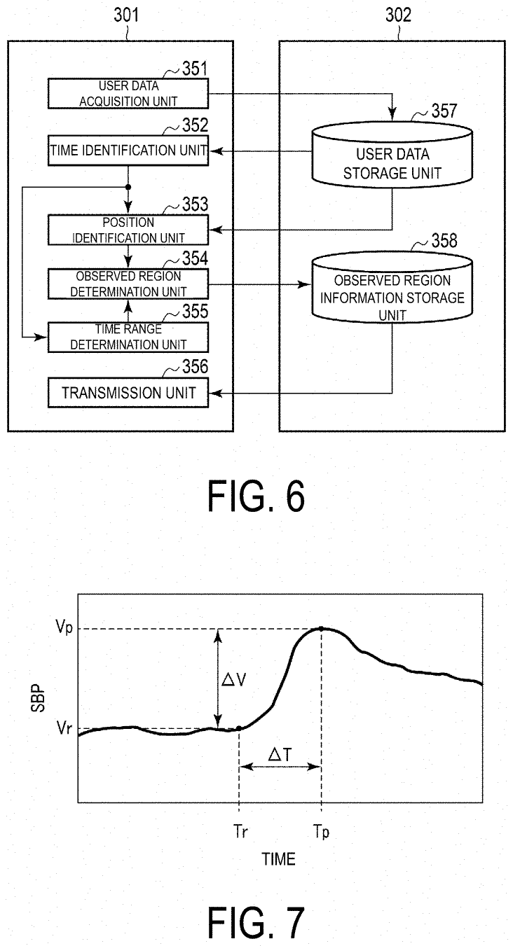

One example of a software configuration of the information processing device 30 according to the present embodiment will be described with reference to FIG. 6. FIG. 6 illustrates one example of the software configuration of the information processing device 30 according to the present embodiment. In the example in FIG. 6, the information processing device 30 includes a user data acquisition unit 351, a time identification unit 352, a position identification unit 353, an observed region determination unit 354, a time range determination unit 355, a transmission unit 356, a user data storage unit 357, and an observed region information storage unit 358. The user data acquisition unit 351, the time identification unit 352, the position identification unit 353, the observed region determination unit 354, the time range determination unit 355, and the transmission unit 356 perform the following processing by executing the observed region determination program stored in the storage unit 302 by the control unit 301 of the information processing device 30. When the control unit 301 executes the observed region determination program, the control unit 301 develops the observed region determination program into the RAM. Then, the control unit 301 interprets and executes the observed region determination program developed into the RAM by the CPU, and controls each component.

The user data storage unit 357 and the observed region information storage unit 358 are provided in the storage unit 302.

The user data acquisition unit 351 acquires user data relating to a plurality of users from the plurality of user terminals UT through the communication interface 304, and stores the acquired user data in the user data storage unit 357. The user data include blood pressure value data and position data.

The time identification unit 352 reads the blood pressure value data from the user data storage unit 357, and identifies, based on the read blood pressure value data, a time when fluctuations in blood pressure value satisfying a predetermined condition have occurred. The time identification unit 352 performs smoothing on the blood pressure value data, for example. For example, the time identification unit 352 identifies a time when a rapid blood pressure rise has occurred, as illustrated in FIG. 7. The rapid blood pressure rise typically occurs for approximately 5 to 20 seconds. This type of the blood pressure rise may occur when, for example, moving from a warm place to a cold place. In this example, the condition mentioned above is a detection condition defined for detecting an occurrence of a rapid blood pressure rise. As one example, the detection condition includes a condition that a blood pressure value rises by greater than or equal to a predetermined value (for example, 10 mmHg) within a predetermined period (for example, 20 seconds). Specifically, the time identification unit 352 detects a peak point (maximum point) from a blood pressure waveform in which the smoothing is performed, and detects a rising point in a time range before the time of the peak point. In this way, the time identification unit 352 acquires a peak blood pressure value Vp, a time Tp indicating the peak blood pressure value Vp, a rising blood pressure value Vr, and a time Tr indicating the rising blood pressure value Vr. Then, the time identification unit 352 detects an occurrence of a rapid blood pressure rise when a difference ΔT acquired by subtracting the time Tr from the time Tp falls within a predetermined time range (for example, a range from 5 seconds to 20 seconds), and a difference ΔV acquired by subtracting the rising blood pressure value Vr from the peak blood pressure value Vp is greater than or equal to a predetermined blood pressure value threshold (for example, 10 mmHg). In this example, when the difference ΔT is less than 5 seconds, it is determined that fluctuations are caused by another factor such as noise. For example, the time identification unit 352 identifies the time Tr indicating a rising blood pressure value as a time when the rapid blood pressure rise has occurred. In another example, the detection condition may be a condition that a blood pressure value exceeds a threshold (for example, 150 mmHg). A condition similar to that mentioned above can also be used even when a physiological indicator is other than a blood pressure value.

The position identification unit 353 identifies, based on the position data, a position where the user has been present at the time identified by the time identification unit 352. The position data include, for example, coordinates (for example, latitude and longitude) acquired at a certain time interval (for example, an interval of one minute). In this case, the position identification unit 353 calculates, base on the position data, coordinates at the time identified by the time identification unit 352 by interpolation such as linear interpolation.

The observed region determination unit 354 determines, as an observed region, a local region including the position identified by the position identification unit 353. For example, the observed region determination unit 354 determines, as the observed region, a circular region having a radius of 50 cm centered in the position identified by the position identification unit 353.

The user data handled by the information processing device 30 include user data relating to a plurality of users, and the user data relating to each of the users include blood pressure value data and position data acquired by long-term observation. Thus, a number of observed regions are determined. The observed region determination unit 354 may combine the observed regions at least partially overlapping each other.

The time range determination unit 355 determines a time range for making a notification for an attention alert, based on the time (for example, date and time) identified by the time identification unit 352. For example, the time range determination unit 355 determines one hour around the time identified by the time identification unit 352 as the time range for making a notification.

The observed region information storage unit 358 stores the observed region information in which the observed region determined by the observed region determination unit 354 and the time range determined by the time range determination unit 355 are associated with each other.

The transmission unit 356 reads the observed region information from the observed region information storage unit 358, and transmits the read observed region information to the user terminal UT via the communication interface 304.

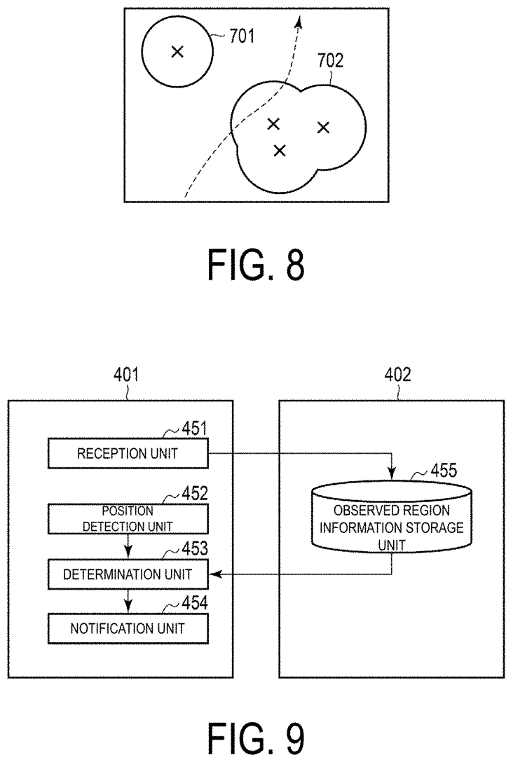

FIG. 8 schematically illustrates a map in which an observed region is set. In FIG. 8, an X mark represents a position where an event has actually occurred, and a solid line surrounding the X mark represents a perimeter of the observed region. An observed region 701 is acquired based on one position in which the event has occurred. An observed region 702 is acquired by combining the observed regions determined based on three positions in which the event has occurred.

### Portable Terminal

One example of a software configuration of the portable terminal 40 according to the present embodiment will be described with reference to FIG. 9. FIG. 9 illustrates one example of the software configuration of the portable terminal 40 according to the present embodiment. In the example in FIG. 9, the portable terminal 40 includes a reception unit 451, a position detection unit 452, a determination unit 453, a notification unit 454, and an observed region information storage unit 455. The reception unit 451, the position detection unit 452, the determination unit 453, and the notification unit 454 perform the following processing by executing the attention alerting program stored in the storage unit 402 by the control unit 401 of the portable terminal 40. When the control unit 401 executes the attention alerting program, the control unit 401 develops the attention alerting program into the RAM. Then, the control unit 401 interprets and executes the attention alerting program developed into the RAM by the CPU, and controls each component. The observed region information storage unit 455 is provided in the storage unit 402.

The reception unit 451 receives observed region information from the information processing device 30 through the communication interface 304, and stores the received observed region information in the observed region information storage unit 455.

The position detection unit 452 detects a position of the portable terminal 40, namely, a position of the target user carrying the portable terminal 40. Specifically, the position detection unit 452 calculates a position of the portable terminal 40, based on a plurality of GPS signals received by the GPS receiver 407. Note that the position detection unit 452 may detect a position of the portable terminal 40 by using a satellite positioning system other than the GPS, such as a global navigation satellite system (GLONASS). Further, the position detection unit 452 may detect a position of the portable terminal 40 by using a satellite positioning system and an indoor positioning system in combination.

The determination unit 453 receives information indicating the position detected by the position detection unit 452, and also reads the observed region information from the observed region information storage unit 455. The determination unit 453 determines whether or not the target user is located in the observed region indicated by the observed region information, based on the position detected by the position detection unit 452. Referring again to FIG. 8, an arrow of a broken line represents a movement track of the target user. In the example in FIG. 8, the target user passes through the observed region 702. When it is detected that the position detected by the position detection unit 452 is located in the observed region 702, the determination unit 453 determines that the target user is located in the observed region 702. The determination unit 453 transmits a signal indicating that the target user has entered the observed region to the notification unit 454.

When receiving the signal indicating that the target user has entered the observed region, the notification unit 454 outputs a notification for an attention alert. The notification can be implemented by any method such as sound, light, and vibration. The notification unit 454 may display a message on the display device of the portable terminal 40. The output of the notification may be transmission of an attention alerting signal to the blood pressure monitor 50 in order to make the notification on the blood pressure monitor 50.

### Blood Pressure Monitor

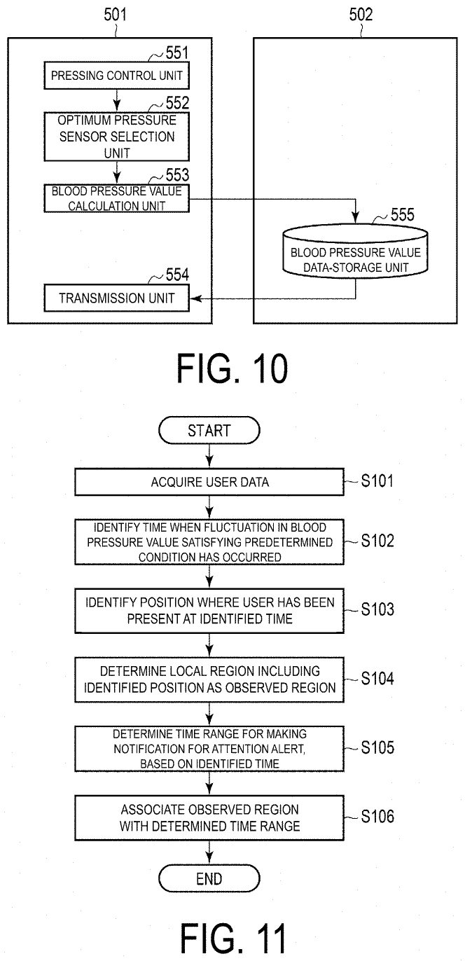

One example of a software configuration of the blood pressure monitor 50 according to the present embodiment will be described with reference to FIG. 10. FIG. 10 illustrates one example of the software configuration of the blood pressure monitor 50. In the example in FIG. 10, the blood pressure monitor 50 includes a pressing control unit 551, an optimum pressure sensor selection unit 552, a blood pressure value calculation unit 553, a transmission unit 554, and a blood pressure value data-storage unit 555. The pressing control unit 551, the optimum pressure sensor selection unit 552, and the blood pressure value calculation unit 553 perform the following processing by executing the blood pressure measurement program stored in the storage unit 502 by the control unit 501 of the blood pressure monitor 50. When the control unit 501 executes the blood pressure measurement program, the control unit 501 develops the blood pressure measurement program into the RAM. Then, the control unit 501 interprets and executes the blood pressure measurement program developed into the RAM by the CPU, and controls each component. The blood pressure value data-storage unit 555 is provided in the storage unit 502.

The pressing control unit 551 controls the pressing portion 507 (FIG. 5). Specifically, the pressing control unit 551 controls the driving of the pump 507B, and the opening and closing of the exhaust valve 507C. The pressing control unit 551 provides a drive signal for driving the pump 507B to the pressing portion 507 in order to supply air to the air bag 507A. The pressing control unit 551 provides a drive signal for opening the exhaust valve 507C to the pressing portion 507 in order to exhaust air from the air bag 507A.

The optimum pressure sensor selection unit 552 selects an optimum pressure sensor from among the pressure sensors of the sensor unit 506. When the sensor unit 506 is pressed against the wrist by the pressing portion 507, a flat portion is generated in the radial artery. A pressure pulse wave detected by the pressure sensor located in this flat portion of the radial artery is not affected by the tension of the wall of the radial artery, and an amplitude becomes the greatest. Further, this pressure pulse wave has the strongest correlation with a blood pressure value. Thus, the optimum pressure sensor selection unit 552 determines the pressure sensor that detects a maximum amplitude of the pressure pulse wave as the optimum pressure sensor. The optimum pressure sensor selection unit 552 provides identification information identifying the pressure sensor selected as the optimum pressure sensor to the blood pressure value calculation unit 553.

The blood pressure value calculation unit 553 receives the identification information from the optimum pressure sensor selection unit 552, and calculates a blood pressure value, based on the pressure signal from the optimum pressure sensor indicated by the identification information. The blood pressure value calculation unit 553 extracts a waveform of the pressure pulse wave of one heart rate, calculates SBP, based on a maximum value in the waveform of the extracted pressure pulse wave, and calculates DBP, based on a minimum value in the waveform of the extracted pressure pulse wave.

The blood pressure value data-storage unit 555 stores blood pressure value data including the blood pressure value for each heart rate. The transmission unit 554 reads the blood pressure value data from the blood pressure value data-storage unit 555, and transmits the read blood pressure value data to the portable terminal 40 through the communication interface 505.

### Other

In the present embodiment, the example is described in which all functions of the information processing device 30, the portable terminal 40, and the blood pressure monitor 50 are achieved by a general-purpose CPU. However, some or all of the above functions may be achieved by one or a plurality of dedicated processors.

### OPERATION EXAMPLE

Next, an operation example of the attention alerting system 20 configured as described above will be described.

First, a processing procedure of determining an observed region will be described.

FIG. 11 illustrates one example of the processing procedure of determining an observed region. In the present embodiment, the processing illustrated in FIG. 11 is performed in the information processing device 30.

In step S101 in FIG. 11, the control unit 301 of the information processing device 30 operates as the user data acquisition unit 351, and acquires user data relating to one or a plurality of users. The user data relating to each user includes blood pressure value data and position data. The processing from step S102 to step S106 below is executed on the individual piece of user data.

In step S102, the control unit 301 operates as the time identification unit 352, and identifies, based on the blood pressure value data included in the user data, a time when fluctuations in the blood pressure value satisfying a predetermined condition have occurred. When a plurality of times are identified, the processing from step S103 to step S106 below is performed on each of the identified times.

In step S103, the control unit 301 operates as the position identification unit 353, and identifies, based on the position data included in the user data, a position where the user has been present at the time identified in step S102.

In step S104, the control unit 301 operates as the observed region determination unit 354, and determines a local region including the position identified in step S103 as an observed region for making a notification for an attention alert.

In step S105, the control unit 301 operates as the time range determination unit 355, and determines a time range for making the notification for the attention alert, based on the time identified in step S102. The observed region determined in step S104 is associated with the time range determined in step S105.

In step S106, the control unit 301 operates as the observed region determination unit 354, and stores, in the observed region information storage unit 455, the observed region information including the observed region determined in step S104 and the time range determined in step S105 in association with each other.

In this way, the observed region for performing the attention alert is determined. Note that the processing procedure mentioned above is merely an example, and the order and the content of the steps may be changed as much as possible. For example, the processing in step S105 may be performed before the processing in step S103 or S104 or simultaneously with the processing of step S103 or S104.

Next, the processing procedure of performing an attention alert will be described.

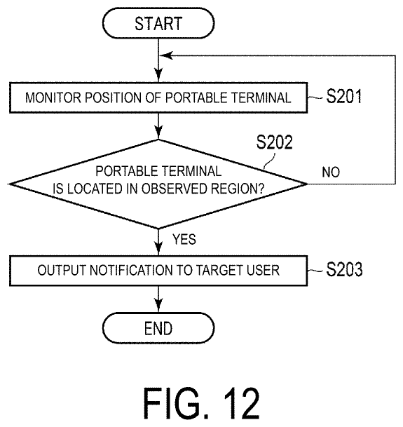

FIG. 12 illustrates one example of the processing procedure of performing an attention alert. In the present embodiment, the processing illustrated in FIG. 12 is performed in the portable terminal 40. The portable terminal 40 receives the observed region information generated as mentioned above relating to FIG. 11 from the information processing device 30.

In step S201 in FIG. 12, the control unit 401 of the portable terminal 40 operates as the position detection unit 452, and monitors a position of the portable terminal 40. In step S202, the control unit 401 operates as the determination unit 453, and determines whether or not the position of the portable terminal 40 is located in the observed region indicated by the observed region information. When the position of the portable terminal 40 is not located in the observed region, the processing returns to step S201. When the position of the portable terminal 40 is located in the observed region, the processing proceeds to step S203. For example, the control unit 401 performs the position detection in step S201 and the determination in step S202 at a certain interval (for example, an interval of 30 seconds), and performs the notification in step S203 when it is determined that the position of the portable terminal 40 is located in the observed region.

In step S203, the control unit 401 operates as the notification unit 454, and notifies the target user that the target user is under a situation where an event is likely to occur. As one example, the control unit 401 outputs a warning sound through the speaker, and also displays, on the display device, a message indicating that the target user is under the situation where the event is likely to occur. In another example, the control unit 401 transmits an attention alerting signal to the blood pressure monitor 50, and the blood pressure monitor 50 receives the attention alerting signal and vibrates, and also displays, on the display device, the message indicating that the target user is under the situation where the event is likely to occur.

Note that the processing procedure mentioned above is merely an example, and the order and the content of the steps may be changed as much as possible.

### Effect

In the embodiment mentioned above, a time when an event (for example, a rapid blood pressure rise) has actually occurred is identified based on blood pressure value data relating to a user, a position where the user has been located at the identified time is identified based on position data, and an observed region and a time range for performing an attention alert relating to the occurrence of the event are determined based on the identified position and the identified time. Then, the position of the target user is monitored, and a notification for the attention alert is output when the target user enters the observed region at the time within the time range. In this way, the target user can be alerted that the target user is under a situation where the event is likely to occur.

User data used for setting an observed region include not only the user data relating to the target user, but also the user data relating to another user. In this way, the observed region is also set in a place where the target user has never visited. As a result, it is also possible to perform an attention alert in a place and the like where the target user visits for the first time.

### MODIFIED EXAMPLES

Note that the present invention is not limited to the embodiment described above.

For example, the user data may further include attribute information representing an attribute of a user. The attribute is, for example, age and/or gender. In this case, the information processing device 30 may determine an observed region for each attribute group associated with the attribute. The attribute group is defined by, for example, age and/or gender. The observed region is associated with the attribute group. In this way, the portable terminal 40 can select the observed region determined based on the user data relating to the user belonging to the same attribute group as that of the target user (the user carrying the portable terminal 40), and make a notification when the target user enters the selected observed region. The event is more likely to occur for the target user in the observed region associated with the user belonging to the same attribute group as that of the target user than in an observed region associated with a user belonging to another attribute group. Therefore, the target user can be alerted when the event is more likely to occur. In other words, the effectiveness of the attention alert is improved.

For example, the user data may further include identification information for identifying a user. In this case, an observed region is associated with the identification information. The portable terminal 40 can identify whether each observed region is associated with the target user or is associated with another user. In this way, an attention alerting method can be changed between the observed region associated with the identification information relating to the target user and the observed region associated with the identification information relating to the another user. The event is more likely to occur for the target user in the observed region associated with the target user than in the observed region associated with the another user. Thus, when the target user enters the observed region associated with the identification information relating to the target user, the effectiveness of the notification can be improved by making a clearer notification (for example, increasing the volume).

For example, the above-mentioned function of the information processing device 30 may be included in each of the user terminals UT. For example, the above-mentioned function of the information processing device 30 may be included in the portable terminal 40, or may be included in the blood pressure monitor 50. When the portable terminal 40 includes the above-mentioned function of the information processing device 30, the portable terminal 40 typically acquires the user data relating to the target user from the blood pressure monitor 50, but may not acquire the user data from the other user terminal UT (specifically, the user terminals UT-2 and UT-3).

For example, each of the user terminals UT may generate the observed region information, based on the user data relating to the target user, and receive the observed region information generated based on the user data relating to another user from the information processing device 30. Specifically, the portable terminal 40 of the user terminal UT-1 may generate the observed region information, based on the user data received from the blood pressure monitor 50, and may further receive the observed region information generated based on the user data relating to the user of the user terminal UT-2 from the information processing device 30.

In short, the present invention is not limited to the embodiment described above as is, and the components can be modified and embodied within a range that does not depart from the gist in a stage of implementation. Further, various inventions can be formed by an appropriate combination of the plurality of components disclosed in the embodiment described above. For example, several components may be deleted from all of the components indicated in the embodiment. Furthermore, components in a different embodiment may be appropriately combined.

### Supplementary Note

A part or all of the present embodiment may also be described as supplementary notes below in addition to claims, which is not limited thereto.

### Supplementary Note 1

An information processing device (11, 30), including:

an acquisition unit (111, 351) configured to acquire user data including physiological indicator data that are measurement data relating to a physiological indicator of a user and position data that are measurement data relating to a position of the user;

a time identification unit (112, 352) configured to identify a time when fluctuations in the physiological indicator satisfying a predetermined condition have occurred, based on the physiological indicator data;

a position identification unit (113, 353) configured to identify a position where the user has been present at the identified time, based on the position data; and

an observed region determination unit (114, 354) configured to determine a local region including the identified position as an observed region for performing an attention alert relating to an occurrence of fluctuations in the physiological indicator.

### Supplementary Note 2

An attention alerting system (10, 20) including an information processing device (11, 30) and a user terminal (12, 40) communicable with the information processing device (11, 30), wherein

the information processing device (11, 30) includes:

an acquisition unit (111, 351) configured to acquire user data including physiological indicator data that are measurement data relating to a physiological indicator of a user and position data that are measurement data relating to a position of the user,

a time identification unit (112, 352) configured to identify a time when fluctuations in the physiological indicator satisfying a predetermined condition have occurred, based on the physiological indicator data,

a position identification unit (113, 353) configured to identify a position where the user has been present at the identified time, based on the position data,

an observed region determination unit (114, 354) configured to determine a local region including the identified position as an observed region for performing an attention alert relating to an occurrence of fluctuations in the physiological indicator, and

a transmission unit (115, 356) configured to transmit observed region information representing the observed region to the user terminal (12, 40), and

the user terminal (12, 40) includes

a reception unit (121, 451) configured to receive the observed region information from the information processing device (11, 30),

a detection unit (122, 452) configured to detect a position of the user terminal (12, 40),

a determination unit (123, 453) configured to determine whether or not the detected position is located in the observed region represented by the received observed region information, and

a notification unit (124, 454) configured to output a notification for the attention alert in response to a determination that the detected position is located in the observed region.

### REFERENCE SIGNS LIST

10, 20 Attention alerting system
UT-1 to 3 User terminal
11 Information processing device
111 User data acquisition unit
112 Time identification unit
113 Position identification unit
114 Observed region determination unit
115 Transmission unit
12 User terminal
121 Reception unit
122 Position detection unit
123 Determination unit
124 Notification unit
30 Information processing device
301 Control unit
302 Storage unit
303 Drive
304 Communication interface
305 External interface
351 User data acquisition unit
352 Time identification unit
353 Position identification unit
354 Observed region determination unit
355 Time range determination unit
356 Transmission unit
357 User data storage unit
358 Observed region information storage unit
40 Portable terminal
401 Control unit
402 Storage unit
403 Input device
404 Output device
405 Communication interface
406 External interface
407 GPS receiver
408 Battery
451 Reception unit
452 Position detection unit
453 Determination unit
454 Notification unit
455 Observed region information storage unit
50 Blood pressure monitor
501 Control unit
502 Storage unit
503 Input device
504 Output device
505 Communication interface
506 Sensor unit
507 Pressing portion
507A Air bag
507B Pump
507C Exhaust valve
508 GPS receiver
509 Battery
551 Pressing control unit
552 Optimum pressure sensor selection unit
553 Blood pressure value calculation unit
554 Transmission unit
555 Blood pressure value data-storage unit
60 Portable terminal
70 Blood pressure monitor
80 Blood pressure monitor

1. An information processing device, comprising:
a processor; and
a memory, wherein
the processor is configured to acquire user data including physiological indicator data that are measurement data relating to a physiological indicator of each of a plurality of users and position data that are measurement data relating to a position of each of the plurality of users;
the processor is configured to identify a time when fluctuations in the physiological indicator satisfying a predetermined condition have occurred, based on the physiological indicator data;
the processor is configured to identify a position where the user in which fluctuations in the physiological indicator satisfying the predetermined condition have occurred has been present at the identified time, based on the position data;
the processor is configured to determine a time range for performing an attention alert relating to an occurrence of fluctuations in the physiological indicator, based on the identified time; and
the processor is configured to determine a local region including the identified position as an observed region for outputting, in a user terminal, a notification for the attention alert when it is detected that the user terminal is located in the local region at a time within the time range.

2. The information processing device according to claim 1, wherein
the user data further include attribute information representing an attribute of each of the plurality of users, and
the processor is configured to associate the observed region with an attribute group based on the attribute.

3. The information processing device according to claim 1, wherein
the user data further include identification information for identifying the plurality of users, and
the processor is configured to associate the observed region with identification information about the user in which fluctuations in the physiological indicator satisfying the predetermined condition have occurred.

4. The information processing device according to claim 1, wherein
the processor is configured to transmit observed region information representing the observed region to a plurality of user terminals, and
the processor is configured to acquire the user data relating to the plurality of users from the plurality of user terminals.

5. The information processing device according to claim 1, wherein
the physiological indicator is a blood pressure value, the condition includes a condition that the blood pressure value rises by greater than or equal to a predetermined value within a predetermined period, and the processor is configured to identify a time when fluctuations in blood pressure value satisfying the condition have occurred.

6. An attention alerting system comprising an information processing device and a user terminal communicable with the information processing device, wherein
the information processing device includes:
a first processor; and
a first memory, wherein
the first processor is configured to acquire user data including physiological indicator data that are measurement data relating to a physiological indicator of each of a plurality of users and position data that are measurement data relating to a position of each of the plurality of users,
the first processor is a time identification unit configured to identify a time when fluctuations in the physiological indicator satisfying a predetermined condition have occurred, based on the physiological indicator data,
the first processor is configured to identify a position where the user in which fluctuations in the physiological indicator satisfying the predetermined condition have occurred has been present at the identified time, based on the position data,
the first processor is configured to determine a time range for performing an attention alert relating to an occurrence of fluctuations in the physiological indicator, based on the identified time;
the first processor is configured to determine a local region including the identified position as an observed region for performing the attention alert, and
the first processor is configured to transmit observed region information representing the observed region to the user terminal, and
the user terminal includes:
a second processor; and
a second memory, wherein
the second processor is configured to receive the observed region information from the information processing device,
the second processor is configured to detect a position of the user terminal,
the second processor is configured to determine whether or not the detected position is located in the observed region represented by the received observed region information, and
the second processor is configured to output, in the user terminal, a notification for the attention alert in response to a determination that the detected position is located in the observed region at a time within the time range.

7. An information processing method executed by a computer, comprising:
acquiring user data including physiological indicator data that are measurement data relating to a physiological indicator of each of a plurality of users and position data that are measurement data relating to a position of each of the plurality of users;
identifying a time when fluctuations in the physiological indicator satisfying a predetermined condition have occurred, based on the physiological indicator data;
identifying a position where the user in which fluctuations in the physiological indicator satisfying the predetermined condition have occurred has been present at the identified time, based on the position data;
determining a time range for performing an attention alert relating to an occurrence of fluctuations in the physiological indicator, based on the identified time; and
determining a local region including the identified position as an observed region for outputting, in a user terminal, a notification for the attention alert when it is detected that the user terminal is located in the local region at a time within the time range.

8. A non-transitory recording medium in which a program is stored for causing the processor included in the information processing device according to claim 1 to acquire, identify, and determine.

9. The information processing device according to claim 2, wherein
the user data further include identification information for identifying the plurality of users, and
the processor is configured to associate the observed region with identification information about the user in which fluctuations in the physiological indicator satisfying the predetermined condition have occurred.

10. The information processing device according to claim 2, wherein
the processor is configured to transmit observed region information representing the observed region to a plurality of user terminals, and
the processor is configured to acquire the user data relating to the plurality of users from the plurality of user terminals.

11. The information processing device according to claim 3, wherein
the processor is configured to transmit observed region information representing the observed region to a plurality of user terminals, and
the processor is configured to acquire the user data relating to the plurality of users from the plurality of user terminals.

12. The information processing device according to claim 2, wherein
the physiological indicator is a blood pressure value, the condition includes a condition that the blood pressure value rises by greater than or equal to a predetermined value within a predetermined period, and the processor is configured to identify a time when fluctuations in blood pressure value satisfying the condition have occurred.

13. The information processing device according to claim 3, wherein
the physiological indicator is a blood pressure value, the condition includes a condition that the blood pressure value rises by greater than or equal to a predetermined value within a predetermined period, and the processor is configured to identify a time when fluctuations in blood pressure value satisfying the condition have occurred.

14. The information processing device according to claim 4, wherein
the physiological indicator is a blood pressure value, the condition includes a condition that the blood pressure value rises by greater than or equal to a predetermined value within a predetermined period, and the processor is configured to identify a time when fluctuations in blood pressure value satisfying the condition have occurred.

15. A non-transitory recording medium in which a program is stored for causing the processor included in the information processing device according to claim 2 to acquire, identify, determine, and associate.

16. A non-transitory recording medium in which a program is stored for causing the processor included in the information processing device according to claim 3 to acquire, identify, determine, and associate.

17. A non-transitory recording medium in which a program is stored for causing the processor included in the information processing device according to claim 4 to acquire, identify, determine, and transmit.

18. A non-transitory recording medium in which a program is stored for causing the processor included in the information processing device according to claim 5 to acquire, identify, and determine.
